(19) United States
(12) Patent Application Publication
(10) Pub. No.: US 2003/0039623 A1
(43) Pub. Date: Feb. 27, 2003

(54) Mono and dialkyl quats with silicone in conditioning compositions

(75) Inventor: Daniel Raymond Pyles, Chicago, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/243,248

(22) Filed: Sep. 13, 2002

Related U.S. Application Data
(63) Continuation of application No. 09/252,564, filed on Feb. 18, 1999.

(51) Int. Cl. A61K 7/06; A61K 7/11; A61K 7/075; A61K 7/08

(52) U.S. Cl. 424/70.12; 424/70.28

(57) ABSTRACT

The present invention relates an opaque or clear conditioner which comprises a monoalkyl quat from C14 to higher carbon chain lengths (preferably C16 to C22) and a dialkyl quat from C14 to higher carbon chain lengths (preferably C16 to C18). Also included is a silicone compound and an emulsifier. Also optionally included is an amount of fatty alcohol necessary to opacify the conditioner. The monoalkyl quat is necessary in a ratio to the dialkyl quat of about 15:1 to about 2:1. The fatty alcohol is optionally present in an amount from about 1% to about 4%.

### BACKGROUND OF THE INVENTION

Most individuals buy and use a hair shampoo for its cleansing properties. In addition to having clean hair, a consumer also desires sufficiently-conditioned hair that holds a preset configuration. However, hair shampoos generally are formulated with highly effective anionic surfactants that primarily clean as opposed to conditioning in the hair. Anionic surfactants not only remove the dirt and soil from the hair, but also remove sebum naturally present on the surface of the hair fibers. Therefore, the desirable cleansing properties of anionic surfactants also leave the hair in a cosmetically-unsatisfactory condition. Shampoos also do not detangle wet hair and do not impart residual conditioning benefits to dry hair, such as manageability or styleability of hair sets.

In general, shampoo compositions containing anionic surfactants, or nonionic surfactants or amphoteric surfactants, leave hair with an undesirable harsh, dull and dry touch, or feel, usually called “creak”, after the hair is shampooed and then rinsed with water. Furthermore, thoroughly cleansed hair also is extremely difficult to comb, in either the wet or the dry state, because the individual hair fibers tend to snarl, kink, and interlock with each other. In addition, incompletely dried hair, such as hair dried with a towel, has poor brushing properties, and after complete drying, the hair does not set well. The combing or brushing property of dry hair remains poor, and the hair has undesirable electrostatic properties in a low humidity atmosphere that causes the hair to “fly away”, thereby further reducing the brushing properties of the hair.

The unsatisfactory combing or brushing property of hair immediately after shampooing, or during trimming treatments after shampooing, also causes hair damage, such as split ends or hair breakage. In addition, the natural luster and resiliency of the hair is reduced. The overall unsatisfactory condition of shampooed hair often necessitates a subsequent post-shampoo treatment of the hair with a conditioning composition to improve these undesirable physical characteristics. Conditioning compositions typically are applied separately from the hair shampoo, and usually are rinses, cream-like emulsions or lotions containing a cationic compound.

Therefore, consumer needs traditionally have been met by the application of a shampoo to cleanse the hair, followed by the application of a conditioner composition to improve wet combing. The commonly accepted method has been to shampoo the hair, followed by rinsing the hair, and then separately applying a conditioner composition, followed by a second rinse. The wet combing problem has been solved by treating shampooed hair with a conditioner composition that coats the hair shaft and causes the individual hair shafts in a tress to resist tangling and matting because of the conditioner residue retained on the shaft.

However, the need for improved compositions that condition the hair, i.e., render the hair more manageable, has long been recognized in the art. As previously discussed, it is well-known that anionic surfactants are suitable for hair shampooing, and that cationic compounds, like cationic surfactants and cationic polymers, are useful as hair conditioners. Therefore, cationic compounds that are substantive to hair often are used to complete the hair cleansing and hair conditioning cycle.

The ability of cationic compounds to adsorb to or interact with the keratinous material of the hair makes these compounds desirable for improving wet hair detangling and dry hair manageability. However, cationic compounds that adsorb particularly strongly to the hair also can reduce the elasticity, body and set of the dried hair. Therefore, although conditioning compositions for application to freshly shampooed hair are well known, new and improved conditioning formulations based on cationic compounds are continually sought.

The following is a list of patents in this field.

JP patents

JP 58032813 A

JP 88002403 B

JP 62126111A

JP 58216111A

JP57056414A

JP57056413A

JP 56169617 A

JP 56169615 A

JP 87008088 B

JP 56169614 A

JP 87008087 B

JP 56169613 A

JP 87008086 B

U.S. patents

U.S. Pat. No. 4,818,523

U.S. Pat. No. 4,976,956.

The present invention is directed to a new opaque or clear conditioning composition that is esthetically acceptable to consumers, improves the wet combing and dry combing properties of hair, and also leaves the dry hair with satisfactory cosmetic and physical properties, including, in particular, dry combing and feel, less hair coating, manageability, body, condition of the ends and set.

### SUMMARY OF THE INVENTION

The invention is an opaque or clear conditioner that has a combination of two different types of conditioning agents, a silicone compound and optionally a fatty alcohol. The present invention is a low solids formulation that provides substantial conditioning benefit without compromising viscosity to users who use conditioners.

The purpose of the invention is to provide a conditioner with improved performance, while using effective materials at ratios that optimize their benefit.

The present invention relates to an opaque or clear conditioner which comprises a monoalkyl quat from C14 to higher Carbon chain lengths (preferably C16 to C22) and a dialkyl quat each alkyl of which is independently from C14 to higher carbon chain lengths (preferably C16 to C22). Also included is an amount of fatty alcohol necessary to opacify the conditioner. Also included is a silicone compound such as an amodimethicone, dimethicone, trimethylsilyl amodimethicone, cyclomethicone or dimethiconol.

Another aspect of the invention is to provide a method of treating the hair to yield well-conditioned hair having esthetically pleasing physical properties by contacting the hair with a conditioner of the present invention.

### DETAILED DESCRIPTION OF THE INVENTION

Unless indicated otherwise, as used herein, % means weight %. The starting materials set forth herein are either known or can be prepared in accordance with known methods.

The present invention relates to an opaque or clear conditioner which comprises a monoalkyl quat from C14 to higher Carbon chain lengths (preferably C16 to C22) and a dialkyl quat each alkyl of which is independently from C14 to higher carbon chain lengths (preferably C16 to C22). Also optionally included is an amount of fatty alcohol necessary to opacify the conditioner. Also included is a silicone compound such as an amodimethicone, dimethicone, trimethylsilylamodimethicone, cyclomethicone or dimethiconol.

Monoalkyl quats can be compounds of the formula N⁺R¹R²R³R⁴X⁻ wherein R¹, R², and R³ are C1-C3 alkyl groups and R⁴ is a C16 or greater alkyl group; and X⁻ is chloride, bromide, methosulfate, ethosulfate, nitrate or tosylate.

Non-limiting examples of monoalkyl quats are:

cetyltrimethylammonium chloride (C16);

stearyltrimethylammonium chloride (C18);

behenetrimethylammonium chloride (C22);

cetrimonium bromide (C16);

soytrimonium chloride (C18);

tallowtrimonium chloride (C18);

behentrimethylammonium methosulfate (C22);

Peg-2 Olealmonium chloride (C18);

palmityltrimethylammonium chloride (C16);

hydrogenated tallowtrimethylammonium chloride (C18);

hydrogenated tallowtrimethylammonium bromide (C18);

hydrogenated tallowtrimethylammonium methosulfate (C18);

cetrimonium tosylate (C16): and

eicosyltrimethylammonium chloride (C20),

Dialkyl quats can be compounds of the formula N⁺R⁵R⁶R⁷R⁸X⁻ wherein R⁵ and R⁶ are C1-C3 alkyl groups and R⁷and R⁸ are C16-C18 alkyl groups; and X⁻ is chloride, bromide, methosulfate, ethosulfate, nitrate, acetate, phosphate; or tosylate.

Non-limiting examples of dialkyl quats are:

dicetyidimethylammonium chloride (C16);

distearyldimethylammonium chloride (C18);

dipalmityldimethylammonium chloride (C16);

dihyrogenatedtallowdimethylammonium chloride (C18);

ditallowdimethylammonium chloride (C18)

dihyrogenatedtallowdimethylammonium bromide (C18)

dihyrogenatedtallowdimethylammonium methosulfate (C18)

Silicone compounds that are used in compositions of the invention include but are not limited to compounds of the formulas:

Amodimethicones

HOSi(CH₃)₂O[SiO(CH₃)₂]x{SiO(OH)[(CH₂)₃NHCH₂CH₂NH₂]}ySi(CH₃)₂OH

where x>20 to 100,000 and y>2 to 20,000

or conforms to the general formula taken from International Cosmetic Ingredient Dictionary Sixth Edition, Vol. 1, page 52 which is hereby incorporated by reference:

HOSi(CH₃)₂OSi[(CH₂)₃NHCH₂CH₂NH₂]OH₂

Nonlimiting examples of amodimethicones are;

Dow Corning 929 (Dow Corning)

Dow Corning 939 (Dow Corning)

Dow Corning 949 (Dow Corning)

GP-4 Silicone Fluid (Genesee)

SM 2059 (General Electric

Wacker Silicone Fluid L 652 (Wacker-Chemie)

E-2153 (Siltech)

Wacker Belsil ADM 6057 E (Wacker-Chemie)

Silicone compounds that are used in compositions of the invention also include but are not limited to compounds of the formulas:

Dimethiconols

HOSi(CH₃)₂O[SiO(CH₃)₂]xSi(CH₃)₂OH

where x>20 or

HOSi(CH₃)₂ O[SiO (CH₃)₂]x{(CH₃)SiO [(CH₃OSi(OCH₃)₂CH₃]}ySi(CH₃)₂OH

where x>10 to 100,000,000 and y>2 to 20,000,000.

Nonlimiting examples of dimethiconols are;

Masil SF-R (Series) (PPG)

Silicone Fluid F-212 (Wacker Silicones)

Silicone Fluid NM 201-50.000 (Huls AG/Huls America)

Silicone Fluid NM 201-2000 (Huls AG/Huls America)

S Series (Siltech)

Abil OSW (Series) (Goldschmidt)

Dow Corning 1401 Fluid (Dow Corning)

Dow Corning 1403 Fluid (Dow Corning)

Dow Corning 2-1870 HVF Emulsion (Dow Corning)

Dow Corning 1784 HVF Emulsion (Dow Corning)

E-2170 (Siltech)

Silatex-D30 (Cosmetic Ingredient Resources/Centerchem)

SM2112 (General Electric)

Wacker Belsil CM 1000 (Wacker-Chemie)

Silicone compounds that are used in compositions of the invention also include but are not limited to:

Dimethicones

which are a mixture of fully methylated linear siloxane polymers end blocked with trimethylsilyloxy units. It conforms to the formula taken from International Cosmetic Ingredient Dictionary Sixth Edition, Vol. 1, page 315 which is hereby incorporated by reference:

(CH₃)₃SiO[Si(CH₃)₂O]xSi(CH₃)₃

x>10 to 100,000.

Nonlimiting examples of dimethicones are;

Abil 10-100000 (Goldschmidt)

Baysilone Fluids M (Miles)

DM Fluid (Series) (Shin Etsu)

Diw Corning 200 Fluid (Dow Corning)

Dow Corning 225 Fluid (Dow Corning)

Masil SF (Series) (PPG)

Mirasil DM (Rhone-Poulenc)

Silicone Fluid NM 1-200 (Huls AG/Huls America)

Silicone Fluid NM 1-350 (Huls AG/Huls America)

Silicone Fluid SWS (Wacker Silicones)

Viscasil (General Electric)

Wacker Belsil DM Grades (Wacker-Chemie)

Wacker Silicone Fluid AKF (Wacker-Chemie)

Abil OSW (Series) (Goldschmidt)

Dow Corning 1669 Cationic Emulsion (Dow Corning)

Dow Corning 1664 Emulsion (Dow Corning)

Dow Corning 593 Fluid (Dow Corning)

Dow Corning 1403 Fluid (Dow Corning)

Silicone compounds that are used in compositions of the invention also include but are not limited to compounds of the formulas:

Cyclomethicones

Technical Names (Cyclohexasilane, dodecamethyl-cyclopentasiloxane, decamethyl-cyclotrisiloxane, hexamethyl-decamethylcyclopentasiloxane, dodecamethylcyclohexasiloxane, and hexamethylcyclotrisiloxane.

Cyclomethicones are cyclic dimethyl polysiloxane compounds that conform generally to the formula taken from International Cosmetic Ingredient Dictionary Sixth Edition, Vol. 1, page 266 which is hereby incorporated by reference:

[(CH₃)₂SiO]ₙ where n averages between 3 and 6.

Nonlimiting examples of cyclomethicones are;

Abil B 8839 (Goldschmidt)

Dow Corning 244 Fluid and 245 Fluid and 344Fluid and 345 Fluid (Dow Corning)

KF994 (Shin Etsu)

KF9945 (Shin Etsu)

Masil SF-V (PPG)

Mirasil CM 4 and 5 (Rhone-Poulenc)

SF-1204 (General Electric)

SF 1202 Fluid (General Electric)

Silicone Fluid SF-1173 (General Electric)

Silicone Fluid NM 4002 and 4004 and 4005 (Huls AG/Huls America)

Siloxane F-222 and 223 and 250 and 251 (Wacker Silicones)

Silicone compounds that are used in compositions of the invention also include but are not limited to compounds of the formulas:

Trimethylsilylamodimethicone is a silicone polymer that conforms to the formula taken from International Cosmetic Ingredient Dictionary Sixth Edition, Vol. 1, page 1046 which is hereby incorporated by reference:

(CH₃)₃[Si(CH₃)₂]ₓ[CH₃Si(CH₂(CHCH₃)CH₂NHCH₂CH₂NH₂]_{y}Si(CH₃)₃

Where x is >20 to 100,000 and y is >2 to 20,000.

Nonlimiting examples of Trimethylsilylamodimethicones are;

Dow Corning 2-8220 Conditioning Additive (Dow Corning)

SF-1708 Silicone Fluid (General Electric)

Silicone Fluid F-801 (Wacker Silicones)

Wacker Silicone Fluid L 653 (Wacker-Chemie)

Dow Corning 7224 Conditioning Agent (Dow Corning)

SM-2115 Silicone Emulsion (General Electric)

Compositions of the invention can contain said silicone compound at from about 0.1% to about 10%; or at from about 0.1% to about 3%; or alternatively at from about 0.25% to about 5%; or more preferably at about; 0.5% to about 3%.

Compositions of the invention can contain said dialkyl quat at from about 0.05% to about 1.5%.

Fatty alcohols are present in opaque compositions of the invention at about 1 to about 4%, more preferably at about 1 to about 2% because at lower fatty alcohol levels there is better silicone deposition from the compositions. Cetyl alcohol is the preferred fatty alcohol.

The following are non-limiting examples of fatty alcohols which may be used in the compositions of the invention:

cetyl alcohol;

stearyl alcohol;

cetearyl alcohol;

behenyl alcohol; and

arachidyl alcohol.

Optional ingredients which may be included in the compositions of the invention are hydrocarbons such as paraffin, vaseline solid paraffin, squalene, oligomer olefins and the like; amidoamines such as stearamidopropyl dimethylamine, isostearamidoethyl morpholine, behenamidopropyl dimethylamine and the like, humectants such as glycerine, propylene glycol, glycerol, sorbitol and the like; esters, such as isopropyl palmitate, isopropyl myristate, and stearyl stearate and the like; emulsifiers such as glyceryl monostearate, sorbitan monopalmitate, polyoxyethylene stearate and the like; cellulose derivatives such as hydroxypropylcellulose; cationic cellulose, hydroxyethyl cellulose and the like; thickening agents such as natural polymers and the like; other silicone compounds such as quaternized silicones, proteinized silicones and the like; and other ingredients such as solvents, bacteriacides, colors, and fragrances.

Compositions of the invention may be prepared by methods which are known to those skilled in the art. Ingredients used in the preparation of compositions of the invention are either known or may be prepared by known methods.

Compositions of the invention are used to condition hair by first wetting the hair, applying the composition of the invention, lathering the hair, and then rinsing the hair. Alternatively, water and conditioner may be applied to the hair simultaneously. Conditioning with compositions may be done right after shampooing when the hair is still wet. Alternatively, conditioning the hair may be done separately from shampooing.

Compositions of the invention provide unexpectedly superior conditioning benefits when compared with prior art formulations. Compositions of the invention unexpectedly provide a high, consumer acceptable viscosity using relatively low levels of monoalkyl quat, dialkyl quat, silicone compounds, and fatty alcohol.

Finally, compositions of the invention provide unexpectedly superior conditioning without the use of increased fatty alcohols.

To demonstrate the new and unexpected results achieved by the present invention, the following compositions were prepared.

As noted above, the present invention contains a monoalkyl quat from C14 to higher carbon chain lengths (preferably C16 to C22) and a dialkyl quat from C14 to higher carbon chain lengths (preferably C16 to C22). Also included is an amount of fatty alcohol necessary to opacify the conditioner. A silicone compound, such as an amodimethicone, dimethicone trimethylsilyl amodimethicone, cyclomethicone, or dimethiconol.

Compositions of the present invention have significantly more conditioning versus a formulation with ingredients that fall outside of the ratios set by the present invention. The following chart illustrates this.

A.) Conditioning Performance.

The present invention is at parity, with regard to conditioning, to the below Control, which is a commercial composition, at a much lower solids level and formula cost.

Instron Wet Combing and Salon Blitz Testing

| Ingredients | A | B | C | D | E | F | Control |
|---|---|---|---|---|---|---|---|
| Cetrimonium Chloride, 30% | 2.8 | 2.8 | 2.8 | 2.8 | 2.8 | 2.8 | — |
| Distearyldimonium Chloride, 95% | .15 | .15 | .15 | .15 | .15 | .15 | — |
| Cetyl Alcohol, 100% | 3 | 3 | 3 | 3 | 3 | 1.8 | 2.75 |
| Natrosol 250 HHR, 97% | — | — | — | — | — | — | .25 |
| Stearamidopropyl | — | — | — | — | — | — | .5 |
| dimethylamine, 99% | | | | | | | |
| Liquid Citric Acid, 50% | — | — | — | — | — | — | .185 |
| Stearyloctyldimonium | — | — | — | — | — | — | 1.75 |
| methosulfate, 83% | | | | | | | |
| Stearyl Alcohol, 100% | — | — | — | — | — | — | 1.25 |
| Behenamidopropyl ethyidimonium | — | — | — | — | — | — | .72 |
| ethosulfate, 35% & | | | | | | | |
| Stearyl alcohol, 65% | | | | | | | |
| DC 949, 34% | — | — | — | — | — | — | 1.25 |
| Cyclomethicone (D4), 100% | — | — | — | — | — | — | 1.6 |
| DC 2-1784, 50% | 2 | — | — | 1 | — | 2 | — |
| DC 2-1870, 25% | — | 4 | — | — | — | — | — |
| DC 949, 34% | — | — | 3.33 | 1.67 | — | — | — |
| Cyclomethicone (DC 245), 100% | — | — | — | — | 1 | — | — |
| Other¹ | Q.S | Q.S. | Q.S | Q.S. | Q.S. | Q.S. | Q.S |
| Instron Wet Combing | | | | | | | |
| Combing Force(gram force) | 12.4bc | 13.3cd | 11.2ab | 11.4ab | 14.2d | 10.4a | 11.3ab |
| Salon Half/Head Testing | | | | | | | |
| Overall Liking | 6.58 | 7.17 | 6.62 | 6.92 | 6.67 | 6.17 | 6.10 |
| Like Conditioning | 6.58ab | 6.83ab | 5.85 b | 6.67ab | 7.33a | 6.33ab | 6.35ab |
| Conditioning | 6.75ab | 6.83ab | 5.69bc | 5.92abc | 7.08a | 5.08c | 6.15abc |
| Softness | 7.50abc | 8.00ab | 7.62abc | 7.58abc | 8.25a | 7.08abc | 7.65abc |
| Moisturized | 6.92ab | 7.00ab | 6.39bc | 6.75abc | 8.00a | 5.92bc | 6.50bc |
| Dryness | 3.00abc | 2.67abc | 4.39a | 3.58abc | 2.33c | 4.25ab | 3.00abc |
| Static | 3.17abc | 2.33bc | 4.31a | 2.75bc | 1.67c | 3.50ab | 2.30bc |

Other¹-water, fragrance, preservatives and other minor ingredients.

Formulas with the same letter are not significantly different from one another at the 90% C.I.

The above chart shows that for Instron Wet Combing several compositions of the invention with various silicones, i.e., compositions A, C, D, and F, are at parity to the control which is a current commercial product. The Instron Wet Combing test is done by standard methods which are known in the art.

The Salon Half/Head results, with an n of approximately 15, show that all of the compositions of the invention with silicones are at parity for Overall Liking versus the control. Several Prototypes have some key conditioning advantages versus the commercial control such as, conditioning, softness, moisturization, less dryness and less static. The compositions of the invention are much lower in cost and contain lower solids than the control.

B.) Silicone Deposition

| Virgin Brown Tresses | ug/g |
|---|---|
| A 1.0% active DC 2-1784 | \|\overline{150} |
| B 1.0% active DC 2-1870 | \|\overline{130} |
| C 1.0% active DC 949 | \|\overline{160} |
| D 0.5% active DC 2-1784 & 0.5% active DC 949 | \|\overline{170} |
| 28 Low Fat Prototype 1 with 1% active DC 2-1784 | \|\overline{440} |

The above chart shows that significant silicone levels (micrograms of silicone, ug, per gram of hair) are deposited using compositions of the invention. The above results on silicone deposition are obtained by using standard methods known in the art.

See previous chart for formula components.

What is claimed is:

1. An opaque or clear hair conditioning composition comprising:
(a) a monoalkyl quat having 14 or greater carbon atoms in an alkyl substituent;
(b) a dialkyl quat having 14 or greater carbon atoms in each alkyl substituent;
(c) a silicone compound; and
(d) optionally a fatty alcohol;

2. A composition according to claim 1 wherein the ratio of (a) to (b) is about 15:1 to about 2:1.

3. A composition according to claim 2 wherein the ratio of (a) to (b) is about 5:1 to 2:1.

4. A composition according to claim 3 wherein the ratio of (a) to (b) is about 5:1 to 3:1.

5. A composition in accordance with claim 1 wherein said fatty alcohol is present in an amount sufficient to opacify the composition.

6. A composition in accordance with claim 1 wherein said fatty alcohol is present at about 1 to about 4%.

7. A composition in accordance with claim 1 wherein said fatty alcohol is present at about 1 to about 2%.

8. A composition in accordance with claim 1 wherein said monoalkyl quat is from C14 to higher carbon chain lengths.

9. A composition in accordance with claim 1 wherein said monoalkyl quat is from C16 to C22.

10. A composition in accordance with claim 1 wherein said monoalkyl quat is a compound of the formula N⁺R¹R²R³R⁴ X⁻
wherein R¹, R², and R³ are C1-C3 alkyl groups and R⁴ is a C16 or greater alkyl group; and X⁻ is chloride, bromide, methosulfate, ethosulfate, nitrate or tosylate.

11. A composition in accordance with claim 1 wherein said monoalkyl quat is selected from the group consisting of
cetyltrimethylammonium chloride;
stearyltrimethylammonium chloride;
behenetrimethylammonium chloride;
cetrimonium bromide;
soytrimonium chloride;
tallowtrimonium chloride;
behentrimethylammonium methosulfate;
Peg-2 Olealmonium chloride;
palmityltrimethylammonium chloride;
hydrogenated tallowtrimethylammonium chloride;
hydrogenated tallowtrimethylammonium bromide;
hydrogenated tallowtrimethylammonium methosulfate;
cetrimonium tosylate; and
eicosyltrimethylammonium chloride.

12. A composition in accordance with claim 1 wherein said monoalkyl quat is selected from the group consisting of behentrimonium chloride and cetrimonium chloride.

13. A composition in accordance with claim 1 wherein said monoalkyl quat is cetrimonium chloride.

14. A composition in accordance with claim 1 wherein each long chain alkyl of said dialkylquat is independently from C14 to higher carbon chain lengths.

15. A composition in accordance with claim 1 wherein each alkyl of said dialkylquat is independently from C16 to C22.

16. A composition in accordance with claim 1 wherein said dialkyl quat is a compound of the formula N⁺R⁵R⁶R⁷R⁸X⁻ wherein R⁵ and R⁶ are C1-C3 alkyl groups and R⁷and R⁸ are C16-C18 alkyl groups; and X⁻ is chloride, bromide, methosulfate, ethosulfate, nitrate, acetate, phosphate; or tosylate.

17. A compound in accordance with claim 1 wherein said dialkyl quat is selected from the group consisting of:
dicetyldimethylammonium chloride;
distearyldimethylammonium chloride;
dipalmityldimethylammonium chloride;
dihyrogenatedtallowdimethylammonium chloride;
ditallowdimethylammonium chloride;
dihyrogenatedtallowdimethylammonium bromide; and
dihyrogenatedtallowdimethylammonium methosulfate.

18. A composition in accordance with claim 1 wherein said dialkyl quat is distearyidimonium chloride.

19. A compound in accordance with claim 1 wherein said silicone compound is an amodimethicone, a dimethicone, a trimethylsilylamodimethicone, cyclomethicone or dimethiconol.

20. A compound in accordance with claim 1 which is an amodimethicone of the formula
HOSi(CH₃)₂O[SiO(CH₃)₂]x{SiO(OH)[(CH₂)₃NHCH₂CH₂NH₂]}ySi(CH₃)₂OH
where x>20 to 100,000 and y>2 to 20,000

21. A compound in accordance with claim 1 which is a dimethiconol of the formula:
HOSi(CH₃)₂O[SiO(CH₃)₂]xSi(CH₃)₂OH
wherein x>20.

22. A compound in accordance with claim 1 which is a dimethiconol of the formula:
HOSi(CH₃)₂O[SiO(CH₃)₂]x{(CH₃)SiO[(CH₃OSi(OCH₃)₂CH₃]}ySi(CH₃)₂OH
where x>10 to 100,000,000 and y>2 to 20,000,000.

23. A compound in accordance with claim 1 which is a dimethicone of the formula
(CH₃)₃SiO[Si(CH₃)₂O]xSi(CH₃)₃
wherein x>10 to 100,000.

24. A compound in accordance with claim 1 which is a cyclomethicone of the cyclic formula [(CH₃)₂SiO]ₙ where n averages between 3 and 6.

25. A compound in accordance with claim 1 which is a trimethylsilylamodimethicone of the formula
(CH₃)₃[Si(CH₃)₂]ₓ[CH₃Si(CH₂(CHCH₃)CH₂NHCH₂CH₂NH₂]_{y}Si(CH₃)₃
wherein x is 20 to 100,000 and y is >2 to 20,000

26. A composition in accordance with claim 1 wherein said composition is selected from the group consisting of:

| Ingredients | A | B | C | D | E | F |
|---|---|---|---|---|---|---|
| Cetrimonium | 2.8 | 2.8 | 2.8 | 2.8 | 2.8 | 2.8 |
| Chloride, 30% | | | | | | |
| Distearyldimonium | .15 | .15 | .15 | .15 | .15 | .15 |
| Chloride, 95% | | | | | | |
| Cetyl Alcohol, 100% | 3 | 3 | 3 | 3 | 3 | 1.8 |
| DC 2-1784, 50% | 2 | — | — | 1 | — | 2 |
| DC 2-1870, 25% | — | 4 | — | — | — | — |
| DC 949, 34% | — | — | 3.33 | 1.67 | — | — |
| Cyclomethicone | — | — | — | — | 1 | — |
| (DC 245), 100% | | | | | | |
| Water, fragrance, and | Q.S. | Q S. | Q S. | Q.S. | Q.S. | Q.S. |
| preservatives | | | | | | |

27. A method for conditioning hair which comprises contacting hair with a composition of claim 1.
